(19) United States
(12) Patent Application Publication
(10) Pub. No.: US 2007/0043236 A1
(43) Pub. Date: Feb. 22, 2007

(54) Process for preparation of gabapentin

(76) Inventors: Thakashina Moorty Chandiran, Tamil Nadu (IN); Jothi Shobana, Tamil Nadu (IN); Kumar Minor Senthil, Tamil Nadu (IN)

Correspondence Address: DUANE MORRIS, LLP;IP DEPARTMENT, 30 SOUTH 17TH STREET, PHILADELPHIA, PA, 19103-4196, US

(21) Appl. No.: 10/558,223

(22) PCT Filed: Jul. 21, 2003
(86) PCT No.: PCT/IN03/00246

§ 371 Date: Sep. 25, 2006

(30) Foreign Application Priority Data
May 19, 2003 (IN) 416/MAS/2003

(51) Int. Cl. C07C 229/20

(52) U.S. Cl. 562/507

(57) ABSTRACT

The present invention relates to the process for the preparation of anhydrous Gabapentin of pharmaceutical grade from the Gabapentin acid addition salts. The process consists of neutralizing the said acid addition salts with an organic base in water to get an aqueous solution comprising of Gabapentin and amine acid addition salt dissolved in water. The process further comprises of a method to separate the Gabapentin and the amine acid addition salt from such an aqueous solution and to recover Gabapentin as an anhydrous Gabapentin form II.

### FIELD OF THE INVENTION

The present invention relates to the process for the preparation of anhydrous Gabapentin of pharmaceutical grade from the Gabapentin acid addition salts. More particularly, the present invention relates to a process for the preparation of anhydrous gabapentin form II from Gabapentin hydrochloride or Gabapentin hydrobromide salt.

### BACKGROUND OF THE INVENTION

Gabapentin is 1-(aminomethyl)cyclohexaneacetic acid having the chemical structure of formula (I).

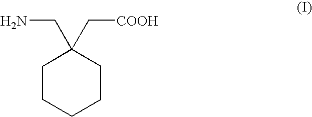

The product was disclosed in the U.S. Pat. No. 4,024,175, and is useful for the therapy of certain cerebral diseases, for example they can be used for the treatment of certain forms of epilepsy, faintness attack, hypokinesia and cranial traumas. This patent also describes the method of preparation of the product from its hydrochloride salt. The hydrochloride salt was treated with basic ion exchanger and then the solvent was evaporated, and the product crystallized from ethanol/ether to obtain anhydrous Gabapentin.

The U.S. Pat. No. 4,894,476 described a method to prepare essentially the same anhydrous Gabapentin described in the U.S. Pat. No. 4,024,175 through pure Gabapentin monohydrate of defined physical characteristics. The process described in this patent comprises of:
a) pouring a solution of an acid salt of 1-(aminomethyl)cyclohexaneacetic acid in deionised water onto an ion exchange column in the basic form and eluting the column with deionized water;
b) concentrating the elute producing a slurry;
c) cooling and adding alcohol to the slurry from step (b);
d) cooling and centrifuging the slurry from step (c);
e) drying the precipitate to obtain Gabapentin monohydrate,
f) The Gabapentin monohydrate was then dissolved in methanol and diluted with isopropyl alcohol to produce a slurry. The slurry was then centrifuged and the precipitate dried to obtain anhydrous Gabapentin.

The European patents EP 1,083,164 and EP 1,174,418 also described the method to prepare the anhydrous Gabapentin from Gabapentin hydrochloride. By the methods described in these patents, hydrochloride salt was treated with basic ion exchange resin and then the solution obtained containing Gabapentin was either concentrated or spray dried to get anhydrous Gabapentin.

The PCT international publication WO 00/01660 described a method to prepare the anhydrous Gabapentin from Gabapentin hydrochloride. The method described in this publication consists of, treating the hydrochloride salt with ion exchange resin, and the aqueous solution thus obtained was concentrated to certain level and the left over water was removed by azeotropic distillation. Further the mass was diluted with isopropyl alcohol and cooled to get anhydrous Gabapentin crystallized from the mass.

Additional methods for the preparation of Gabapentin were also described in the US patent applications US 2002/0061931 and US 2002/0045662 (equivalent to PCT application nos. WO01/97612 and WO01/97782), which described a process wherein gabapentin HCl was, dissolved in dry isopropanol and activated carbon was added. The suspension was heated under predetermined parameters and was washed with isopropanol. After sometime, tributylamine was added and the gabapentin base formed was separated from the suspension by filtration and washed with methanol.

Another PCT international publication WO 00/58268 described a method to prepare the anhydrous Gabapentin from Gabapentin hydrochloride. The method described in this publication consisted of, treating the hydrochloride salt aqueous solution with a base to Gabapentin isoelectric point and then diafiltering the solution through highly selective membrane to separate chloride ion and Gabapentin substantially free from chloride ions.

The PCT international publication WO 02/34709 described a process for the purification of Gabapentin hydrochloride from the inorganic salts and its conversion into Gabapentin by treatment of an aqueous Gabapentin hydrochloride solution with a cation exchange resin.

Alternative ways of preparing anhydrous Gabapentin from hydrochloride was disclosed in U.S. Pat. No. 6,255,526 (equivalent to WO98/28255). The described method in this patent consisted of first pre-treating Gabapentin HCl with a second solvent to remove inorganic salts, then dissolving it in a first solvent and subsequently treating with an amine to form a precipitate. This anhydrous Gabapentin had physical characteristics different from that of the product known to be used in the pharmaceutical preparations. The new anhydrous polymorph obtained by this process was designated as form III. This form III was then crystallized with Methanol to give gabapentin form II.

The PCT international publication WO 02/44123 described a separation process of Gabapentin form II starting with Gabapentin hydrochloride in which the chloride ion was eliminated by precipitating it as an insoluble salt, releasing Gabapentin in aqueous solution as free amino acid. The aqueous solution on further evaporation under reduced pressure and treated with alcohol to get Gabapentin form II.

Most of the described methods involve the use of ion exchange column and thus require the use of large amount of ion-exchanger for a longer period of time so as to lower the level of chloride ion.

Further, alternative methods for preparing gabapentin have been described that do not proceed via the hydrochloride or other mineral acid salt. Such methods include those described in U.S. Pat. Nos. 5,132,451; 5,095,148; 5,068,413. These methods disclosed in these patents involve a lot more demanding steps and industrially impractical.

Further the described processes obtain gabapentin form II through gabapentin Form III.

### SUMMARY OF THE INVENTION

The object of the present invention was therefore to develop a simple and viable alternate method to produce anhydrous Gabapentin form II, starting from Gabapentin acid addition salts.

To achieve the said object the present invention provides a novel method to prepare anhydrous Gabapentin form II from Gabapentin acid addition salts such as Gabapentin hydrochloride and Gabapentin hydrobromide. The process consists of neutralizing the said acid addition salts with an organic base in water to get an aqueous solution comprising of Gabapentin and amine acid addition salt dissolved in water. The process further comprises of a method to separate the Gabapentin and the amine acid addition salt from such an aqueous solution and to recover Gabapentin as an anhydrous Gabapentin form II.

### DETAILED DESCRIPTION OF THE INVENTION

The preferred embodiment of the present invention provides a method for preparing anhydrous gabapentin form II comprising: (a) dissolution of Gabapentin acid addition salt in water, (b) neutralization of the acid addition salt with a base to get a aqueous solution comprising of Gabapentin and amine acid addition salt dissolved in water, (c) distillation of water from the solution obtained in step (b) under reduced pressure, (d) seeding the concentrate with Gabapentin form II at an appropriate instance during said distillation to initiate the crystallization of Gabapentin form II while the solution is being concentrated, (e) addition of alcohol or mixture of alcohols in to the concentrate to enable the amine acid addition salt dissolved in it, (f) recovery of the crystallized Gabapentin form II from the mixture by filtration and (g) purification of the product by recrystallization from a solvent.

The Gabapentin acid addition salt in step (a) is inorganic acid addition salt such as hydrochloride or hydrobromide salts of gabapentin and like. The said acid addition salt is dissolved in water to get an aqueous solution. The dissolution is preferably carried out with 2 to 5 times the weight of water in proportion to the Gabapentin acid addition salt. The dissolution is carried out at a temperature between 20 to 50° C. and preferably at 25 to 30° C.

The neutralization step (b) is carried out with base, preferably with organic bases, such as amines. The preferred amines are primary, secondary and tertiary alkyl amines. The most preferred amines are triethylamine, tripropylamine and tributylamine. For neutralization 0.9 to 1.2 mole equivalence of amines is used, preferably 0.95 to 1.05 mole equivalence of amine is used for the neutralization. The pH of the solution is 6.5 to 7.5 after the neutralization and the preferred pH of neutralization is 6.8 to 7.2. The neutralization step is carried out at a temperature ranging between 10-50° C., preferably at a temperature ranging between 20-30° C. to get an aqueous solution comprising of Gabapentin and amine acid addition salt dissolved in water.

In step-c water is removed from the aqueous solution by distillation under reduced pressure. Water is distilled at 30 to 70° C. under reduced pressure, preferably at 45 to 55° C. In step (d); during the water distillation, Gabapentin from II seeds are added after distilling out 20 to 50% of water quantity used for the dissolution of the Gabapentin acid addition salt. How ever the appropriate seeding instance could vary with the temperature of the distillation and quantity of the water used in the process. The addition of Gabapentin form II seeds at this instance initiates the crystallization of Gabapentin form II during the water distillation. The Gabapentin form II seeds added are about 1 to 5 g for every 100 g of Gabapentin acid addition salt and preferably 2 g seeds for every 100 g of Gabapentin acid addition salt used in the process. Water distillation is continued till 80 to 95% quantity of water used for the dissolution of the Gabapentin acid addition salt is distilled out. A small sample is removed from the concentrate at this point and filtered, washed with chilled water and dried under vacuum. Examined the product obtained by IR (KBr) and X-ray diffraction, and it is identified to match with those recorded from Gabapentin form II.

The concentrate is then diluted with alcohol or with mixture of alcohols in step (e) and then cooled to 0 to 20° C. The dilution with alcoholic solvent facilitates the dissolution of the amine acid addition salts in the solvent and removal from the concentrate so as to enable the separation of Gabapentin form the amine acid addition salts. The preferred alcohols are methyl alcohol, ethyl alcohol, isopropyl alcohol and butyl alcohol or their mixture of one in another. The preferred alcohol mixture ia a mixture of methyl alcohol and isopropyl alcohol.

The slurry obtained from step (e) is then filter and washed with the solvent to get the product Gabapentin form II. A sample from the product is again re-examined by IR (KBr) and X-ray diffraction and it is identified to match with those recorded from Gabapentin form II.

The product is then recrystallized from methyl alcohol or ethyl alcohol to obtain a pure product with anion content less than 100 ppm by silver nitrate solution titration or by ion chromatography and lactam impurity of formula (II) content less than 0.1% wt/wt by HPLC analysis.

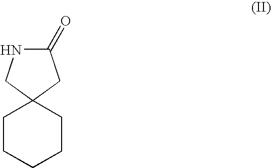

The most preferred method for the preparation of anhydrous Gabapentin form II is described as follows:

Gabapentin hydrobromide is dissolved in twice the weight of water at 25 to 30° C. to get an aqueous solution. The aqueous solution is then neutralized with tributylamine at 25 to 30° C. over a period of 30 min. The solution is then stirred at 25 to 30° C. over a period of 30 min. The aqueous solution is then heated to 45 to 50° C. and distilled out 35% of water under reduced pressure. Added Gabapentin form II seed crystals to the solution and continued the water distillation to the extent of around 90% of the total water used for the dissolution of the gabapentin salt in the process. The concentrate is then diluted with a mixture of methyl alcohol and isopropyl alcohol. The slurry so obtained is then cooled to 0 to 5° C. and filtered. The product is then recrystallized from methyl alcohol.

The present invention will now be described in more detail by way of examples, which should not be construed as limiting the invention thereto.

### EXAMPLE 1

Preparation of Gabapentin from Gabapentin Hydrobromide

Gabapentin hydrobromide (200 g, 0.793 mole) was dissolved in water (400 ml) at 30° C. Into this solution was added tributylamine (154 g, 0.83 mole) at 25-30° C. The p^{H} of the solution was 6.9 at the end of tributylamine addition. The solution was then warmed to 50° C. and distilled out water at 45 to 50° C. at 35 to 40 mm Hg. Added in to the content, Gabapentin form II seeds (4 g) when water (160 ml) was distilled out from the solution. Continued the distillation at 45 to 50° C. at 35 to 34 mm Hg till water (360 ml) was collected. Removed small from the slurry after the water distillation and filtered, washed with chilled water and dried at 40-45° C. and 10 mmHg and examined the sample by IR (KBr) and X-Ray Diffraction. The sample IR (KBr) and X-Ray diffraction results matched with those recorded from Gabapentin form II. Added in to the slurry a mixture of isopropyl alcohol (360 ml) and methanol (240 ml) and the content was cooled to 0-5° C. and maintained at this temperature for 6 hrs. The content was then filtered, washed with a mixture of isopropyl alcohol (240 ml) and methanol (160 ml) and then with acetone (400 ml) to afford crude Gabapentin form II 96 g (Yield; 70.8%, IR (KBr) and X-Ray Diffraction examinations matched with those recorded from Gabapentin form II).

### EXAMPLE 2

Preparation of Gabapentin from Gabapentin Hydrobromide

Gabapentin hydrobromide (200 g, 0.793 mole) was dissolved in water (400 ml) at 30° C. Into this solution was added tributylamine (154 g, 0.83 mole) at 25-30° C. The p^{H} of the solution was 6.9 at the end of tributylamine addition. The solution was then warmed to 50° C. and distilled out water at 45 to 50° C. at 35 to 40 mm Hg. Added in to the content Gabapentin form II seeds (4 g) when water (140 ml) was distilled out. Continued the distillation at 45 to 50° C. at 35 to 34 mm Hg till water (360 ml) was collected. Removed small from the slurry after the water distillation and filtered, washed with chilled water and dried at 40-45° C. and 10 mmHg and examined the sample by IR (KBr) and X-Ray Diffraction. The sample IR (KBr) and X-Ray diffraction results matched with those recorded from Gabapentin form II. Added in to the slurry a mixture of isopropyl alcohol (600 ml) and methanol (400 ml) and the content was cooled to 0-5° C. and maintained at this temperature for 6 hrs. The content was then filtered, washed with a mixture of isopropyl alcohol (240 ml) and methanol (160 ml) and then with acetone (400 ml) to afford crude Gabapentin form II 99 g (Yield; 73.0%, IR (KBr) and X-Ray Diffraction examinations matched with those recorded from Gabapentin form II).

### EXAMPLE 3

Preparation of Gabapentin from Gabapentin Hydrobromide

Gabapentin hydrobromide (50 g, 0.198 mole) was dissolved in water (100 ml) at 30° C. Into this solution was added tributylamine (38.5 g, 0.207 mole) at 25-30° C. The p^{H} of the solution was 6.8 at the end of tributylamine addition. The solution was then warmed to 50° C. and distilled out water at 45 to 50° C. at 35 to 40 mm Hg. Added in to the content Gabapentin form II seeds (1 g) when water (35 ml) was distilled out. The distillation was continued till water (85 ml) was collected. The slurry was cooled to 30° C. and filtered, the product washed with pre cooled water (25 ml) and product dried at 40-45° C. at 10 mmHg to afford Gabapentin form II 22 g. (Yield; 65%, IR (KBr) and X-Ray Diffraction examinations matched with those recorded from Gabapentin form II).

### EXAMPLE 4

Preparation of Gabapentin from Gabapentin Hydrobromide

Gabapentin hydrobromide (100 g, 0.396 mole) was dissolved in water (200 ml) at 30° C. Into this solution was added tributylamine (77 g, 0.415 mole) at 25-30° C. The p^{H} of the solution was 7.13 at the end of tributylamine addition. The solution was then warmed to 50° C. and distilled out water at 45 to 50° C. at 35 to 40 mm Hg. Added in to the content Gabapentin form II seeds (2 g) when Water (100 ml) was distilled out. Continued the distillation at 45 to 50° C. at 35 to 34 mm Hg till water (180 ml) was collected. Removed small from the slurry after the water distillation and filtered, washed with chilled water and dried at 40-45° C. and 10 mmHg and examined the sample by IR (KBr) and X-Ray Diffraction. The sample IR (KBr) and X-Ray diffraction results matched with those recorded from Gabapentin form II. Added in to the slurry a mixture of isopropyl alcohol (180 ml) and methanol (120 ml) and the content was cooled to 0-5° C. and maintained at this temperature for 4 hrs. The content was then filtered, washed with a mixture of isopropyl alcohol (120 ml) and methanol (80 ml) and then with acetone (200 ml) to afford crude Gabapentin form II 43 g (Yield; 64%, IR (KBr) and X-Ray Diffraction examinations matched with those recorded from Gabapentin form II).

We claim:

1. A process for conversion of an aqueous solution for gabapentin acid addition salt into gabapentin form II without the isolation of gabapentin form III comprising the steps of:
a neutralizing of the aqueous solution of gabapentin acid addition salt with a base
b. addition of seed crystals of gabapentin form II
c. distillation of aqueous solvent to obtain a concentrate
d. dilution of the concentrate with a second solvent to obtain a slurry and
e. filtering said slurry to obtain gabapentin form II

2. A process as claimed in claim 1 wherein said gabapentin acid addition salt is an inorganic acid addition salt

3. A process as claimed in claim 2 wherein said inorganic gabapentin acid addition salt is a hydrochloride or hydrobromide salt of gabapentin.

4. A process as claimed in claim 1 wherein said solvent for dissolution of said gabapentin acid addition salt is water.

5. A process as claimed in claim 1 wherein said organic base is an amine.

6. A process as claimed in claim 5 wherein said amine is a primary, secondary or tertiary amine.

7. A process as claimed in claim 6 wherein said amine is selected from triethyl amine, tributylamine and tripropylamine.

8. A process as claimed in claim 5 wherein 0.9 to 1.2 mole equivalent of said amine is used for neutralization.

9. A process as claimed in claim 8 wherein 0.95 to 1.05 mole equivalent of said amine is used for neutralization.

10. A process as claimed in claim 1 wherein the pH of the solution after neutralization is 6.5 to 7.5

11. A process as claimed in claim 1 wherein said distillation of said solvent is carried out at a temperature between 30-70° C. under reduced pressure.

12. A process as claimed in claim 11 wherein said distillation of said solvent is carried out at a temperature between 45-55° C. under reduced pressure.

13. A process as claimed in claim 1 wherein the amount of said gabapentin form II seeds added in step c) is 1-5% by weight of said gabapentin acid addition salt.

14. A process as claimed in claim 13 wherein the amount of said gabapentin form II seeds added in step c) is 2% by weight of said gabapentin acid addition salt.

15. A process as claimed in claim 1 wherein said second solvent is an alcohol

16. A process as claimed in claim 15 wherein said alcohol is selected from methyl alcohol, ethyl alcohol isopropyl alcohol, butyl alcohol or mixtures thereof

17. A process as claimed in claim 16 wherein said alcohol mixture is a mixture of methyl alcohol and isopropyl alcohol.

18. A process as claimed in claim in claim 1 further including the step of subjecting gabapentin form II to purification step of recrystallization.

19. A process as claimed in claim 18 wherein said recrystallization is done from methyl alcohol, ethyl alcohol or mixture of methyl alcohol and isopropyl alcohol.

20. A process for preparing anhydrous gabapentin form II substantially as herein described with reference to the foregoing examples.

21. A process for converting an aqueous solution of gabapentin hydrochloride into gabapentin form II without the isolation of gabapentin form III.
